(19) United States
(12) Patent Application Publication
(10) Pub. No.: US 2025/0197846 A1
(43) Pub. Date: Jun. 19, 2025

(54) TAGMENTATION WORKFLOW

(71) Applicant: ILLUMINA, INC., San Diego, CA (US)

(72) Inventors: Karolina Kuna, Cambridge (GB); Natalie Morrell, Cambridge (GB); Niall Anthony Gormley, Cambridge (GB); Andrew Slatter, Cambridge (GB); Carole Anastasi, Cambridge (GB)

(21) Appl. No.: 18/851,535

(22) PCT Filed: Jul. 27, 2023
(86) PCT No.: PCT/EP2023/070845

§ 371 Date: Sep. 26, 2024

Related U.S. Application Data
(60) Provisional application No. 63/392,729, filed on Jul. 27, 2022.

(51) Int. Cl. C12N 15/10; C12Q 1/44; C12Q 1/6806; C12Q 1/6853

(52) U.S. Cl. CPC C12N 15/1065; C12Q 1/44; C12Q 1/6806; C12Q 1/6853; G01N 2333/922

(57) ABSTRACT

In an example of a method, a deoxyribonucleic acid sample is exposed to tagmentation in the presence of a tagmentation buffer including a divalent cation cofactor and a transposase enzyme to generate a tagmented DNA fragment complex. A chelator mixture is added to the tagmented DNA fragment complex. The chelator mixture includes a chelator of the divalent cation cofactor at a weight ratio that is at least 1:1 with the divalent cation cofactor; and has a pH ranging from 8 to 9. The tagmented DNA fragment complex is incubated in the chelator mixture at a temperature of at least 55° C. for at least about 60 seconds, so that the transposase enzyme dissociates from a tagmented DNA fragment of the tagmented DNA fragment complex.

### CROSS-REFERENCE TO RELATED APPLICATION

This application is a national stage entry under 35 U.S.C. § 371 of PCT/EP2023/070845, filed Jul. 27, 2023, which itself claims the benefit of U.S. Provisional Application Ser. No. 63/392,729, filed Jul. 27, 2022, the contents of each of which is incorporated by reference herein in its entirety.

### BACKGROUND

Double-stranded deoxyribonucleic acid (dsDNA) target molecules can be fragmented and tagged to generate a library of smaller, double-stranded DNA molecules. The smaller double-stranded DNA molecules can be denatured to generate small single-stranded DNA molecules (ssDNA). These small, single-stranded DNA molecules may be used as templates in DNA sequencing reactions. The templates may enable short read lengths to be obtained, and then during data analysis, overlapping short sequence reads can be aligned to reconstruct the longer nucleic acid sequences. Some methods for fragmentation and tagging of double-stranded DNA generate excessive waste, involve expensive instruments for fragmentation, and are time-consuming.

### SUMMARY

The methods disclosed herein involve tagmentation, which is followed by the dissociation and removal of the transposase enzyme that is used in tagmentation. The transposase enzyme is dissociated with a chelator of the divalent cation cofactor that is used during tagmentation. Thus, the methods are performed without a chaotropic agent; the presence of which can denature enzymes (e.g., ligase, polymerase) used downstream of the tagmentation process.

### BRIEF DESCRIPTION OF THE DRAWINGS

Features of examples of the present disclosure will become apparent by reference to the following detailed description and drawings, in which like reference numerals correspond to similar, though perhaps not identical, components. For the sake of brevity, reference numerals or features having a previously described function may or may not be described in connection with other drawings in which they appear.

FIG. 1 is a schematic diagram illustrating one example of tagmentation, illustrating how a transposome complex fragments DNA and appends adaptors by covalently linking a transferred strand to the 5′ ends of each of the double stranded fragment strands and how the 3′ ends of the DNA fragment strands remain unlinked;

FIG. 2 is a schematic diagram illustrating a process following tagmentation where a polymerase is used to append, by extension, adaptor sequences to the 3′ ends of fragment strands in a tagmented molecule;

FIG. 3A is a schematic diagram of transposome complexes including a forked adapter;

FIG. 3B is a schematic diagram illustrating a process following tagmentation where a ligase and a non-strand-displacing polymerase are used to append, by extension and ligation, adaptor sequences, provided by the non-transferred strand of the transposome complex of FIG. 3A, to the 3′ ends of fragment strands in a tagmented molecule;

FIG. 4 is a graph depicting the DNA yield (Y axis, normalized fluorescence units (FU)) versus the insert size (X axis, number of base pairs (bp)) from two different comparative methods for removing the Tn5 transposase enzyme after tagmentation;

FIG. 5 is a graph depicting the DNA yield (Y axis, normalized fluorescence units (FU)) versus the insert size (X axis, number of base pairs (bp)) from one comparative method (A) and one example method (B) for removing the Tn5 transposase enzyme after tagmentation;

FIG. 6 is a graph depicting the DNA yield (Y axis, normalized fluorescence units (FU*103)) versus the insert size (X axis, number of base pairs (bp)) from a replicated comparative method (A and B) and a replicated example method (C and D) for removing the Tn5 transposase enzyme after tagmentation;

FIG. 7 is a graph depicting the DNA yield (Y axis, normalized fluorescence units (FU)) versus the insert size (X axis, number of base pairs (bp)) from one comparative method (A) and one example method (B) for removing the Tn5 transposase enzyme after tagmentation;

FIG. 8 is a graph depicting the DNA yield (Y axis, normalized fluorescence units (FU)) versus the insert size (X axis, number of base pairs (bp)) from a positive control method (A), a first comparative method (B), an example method (C), and a second comparative method (D) for removing the Tn5 transposase enzyme after tagmentation;

FIG. 9 is a graph depicting the DNA yield (Y axis, normalized fluorescence units (FU)) versus the insert size (X axis, number of base pairs (bp)) from an example method (A), positive control method (B), and a comparative method (C) for removing the Tn5 transposase enzyme after tagmentation;

FIG. 10 is a graph depicting the DNA yield (Y axis, normalized fluorescence units (FU)) versus the insert size (X axis, number of base pairs (bp)) for a control process (lines A and B) and for an example process (lines C and D);

FIG. 11 is a graph depicting the callability (Y axis, ×100=%) of a sequenced control sample and a sequenced example sample across different regions of the human genome with different AT and GC contents; and

FIG. 12A through FIG. 12D are graphs depicting the single nucleotide polymorphism (SNP) recall (×100=%, Y axis, FIG. 12A), the SNP precision (×100=%, Y axis, FIG. 12B), the insertion-deletion mutation (indel) recall (×100=%, Y axis, FIG. 12C), and the indel precision (×100=%, Y axis, FIG. 12D), for sequenced control sample and the sequenced example sample.

### DETAILED DESCRIPTION

Tagmentation is a process in which a deoxyribonucleic acid (DNA) sample is cleaved/fragmented and tagged (e.g., with the adapters) for analysis. Tagmentation is an in vitro transposition reaction.

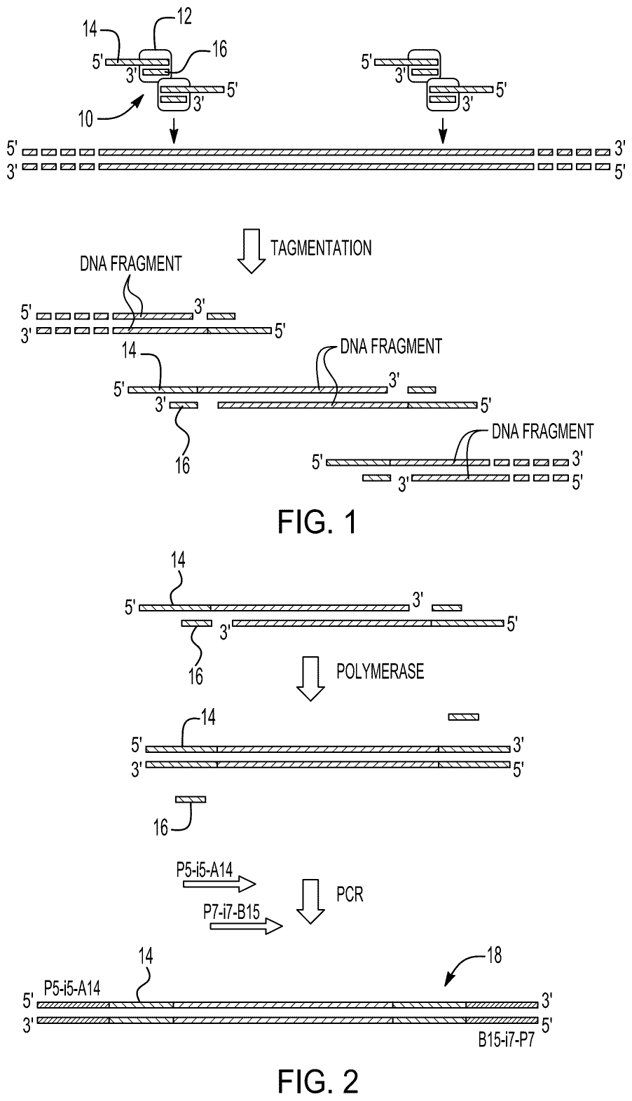

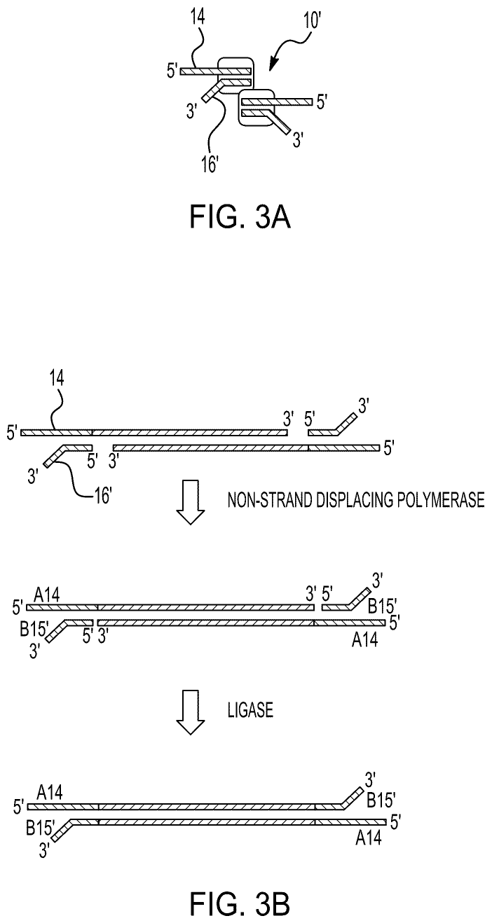

In the example shown in FIG. 1, tagmentation relies on a transposase enzyme 12 (e.g., Tn5) that fragments and simultaneously appends adaptor sequences to the 5′ ends of double stranded DNA fragments. The transposase enzyme 12 is part of a transposome complex 10. The transposome complex 10 includes the transposase enzyme 12 non-covalently bound to a transposon end. Each transposon end is a double-stranded nucleic acid strand, one strand of which is part of a transferred strand 14 and the other strand of which is part of a non-transferred strand 16 (FIGS. 1 and 2) or 16′ (FIGS. 3A and 3B). In other words, the transposon end includes a portion of the transferred strand 14 that is hybridized to a portion of the non-transferred strand 16, 16′. As examples, the transposon ends may be the related but non-identical 19-base pair (bp) outer end and inner end sequences that serve as the substrate for the activity of the Tn5 transposase 12, or the mosaic ends recognized by a wild-type or mutant Tn5 transposase, or the R1 end and the R2 end recognized by the MuA transposase.

The tagmentation process generates a tagmentation complex (also referred to herein as “tagmented DNA fragment complex”), which includes the transposase enzyme 12 bound to the tagmented DNA.

The tagmentation process is followed by one or more additional step(s) that is/are independent of the transposase enzyme 12. In this step or these steps, an adapter (which may be similar to or different from the adaptor added to the 5′ ends during tagmentation) is appended to the 3′ ends of the fragments. The 3′ end adaptors may be added by either of the examples shown in FIG. 2 or FIG. 3B (described in more detail below). In the example shown in FIG. 2, the adapters that are added include those for clustering (e.g., P5, P7), indexing (i5, i7), and sequencing primer binding sites (A14, B15). As such, the library template 18 generated via the example shown in FIG. 2 is in a form ready for flow-cell clustering and sequencing. In the example shown in FIG. 3B, sequencing primer binding sites (A14, B15) are added first, and the generated library template 18′ can be exposed to additional processing to add adapter sequences for clustering and sequencing and indexing sequences (e.g., barcode sequences). Alternatively, the forked adaptor shown in FIG. 3A and FIG. 3B may instead include all of the desired sequences (e.g., P5-15-A14 and P7-i7-B15) as part of the non-transferred strand 16′ of the transposome complex 10′.

As mentioned, one example of the addition of the 3′ end adaptors is shown in FIG. 2. In this example, the free 3′ end of the fragment can be extended in the presence of a polymerase, deoxyribonucleoside triphosphates (dNTPs), and heat to remove the non-transferred strands 16; and then the complement of the 5′ adaptor (i.e., the transferred strand 14) is copied; and finally a polymerase chain reaction (PCR) reaction with two distinct primers can be used to enrich the primary tagmentation molecules so that they have a P5 based adaptor on one end and a P7 based adaptor on the other end. In the example shown in FIG. 3, the two distinct primers are P5-15-A14 and P7-i7-B15, each of which includes an adapter sequence for clustering and sequencing (P5, P7), an indexing sequence (i5, i7), and a sequencing primer binding site (A14, B15)).

Another example of the addition of the 3′ end adaptors is shown in FIG. 3B. The transposome complex 10′ used in this example method includes a single double-stranded forked adaptor, which is shown in FIG. 3A. In the method of FIG. 3B, a non-displacing polymerase is used at a temperature below the primer melting temperature (Tm) of the non-transferred strand 16′, to extend the free 3′ end of the fragment until it reaches the 5′ end of the non-transferred adaptor strand 16′ and then a ligase covalently connects the non-transferred strand 16′ to the fragment.

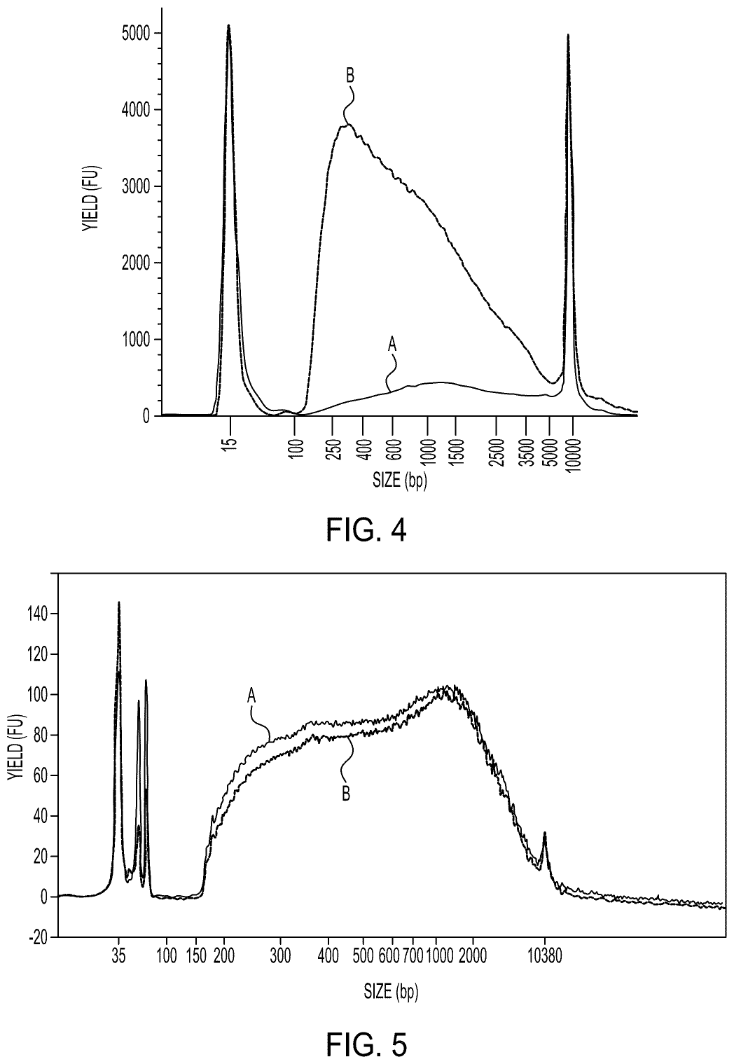

In any example of tagmentation, the transposase enzyme 12 is removed from the tagmentation complex (which includes the transposase enzyme 12 bound to the tagmented DNA fragment) before the examples shown in FIG. 2 and FIG. 3B can be used to add adaptors to the 3′ end of the tagmented DNA. This is because the transposase enzyme 12, especially Tn5, remains tightly bound to the tagmented DNA and inhibits enzymes used to complete the addition of the 3′ adaptor from accessing the DNA. Heating the tagmentation complex to high temperatures, such as those used in PCR that are designed to append adaptor sequences to the 3′ end, is only partially effective, as evidenced by the reduced yield and larger insert sizes in prepared libraries (see FIG. 4, line A, described in more detail in the Example section). Complete Tn5 displacement may be achieved by adding a strong denaturant or chaotropic agent, such as sodium dodecyl sulfate (SDS), to the tagmentation complex to denature and unravel the Tn5 protein and fully dissociate it from the tagmented DNA. This method is more effective than heating alone, as evidence by the data shown in FIG. 4, line B. However, the strong denaturant can denature enzymes (e.g., ligase, polymerase) that are used in the downstream processes, such as extension reactions and amplification reactions. As such, the denaturant should be thoroughly washed away in order to prevent the inhibition of subsequent enzymatic steps.

The examples set forth herein provide an alternative method to remove the transposase enzyme (e.g., the Tn5 protein) from its tagmentation complex that does not involve a denaturant, and thus is compatible with downstream enzymatic steps. This method simplifies library preparation, e.g., Tn5 library preparation, workflows by removing the strong denaturant(s) and washes.

More specifically, the method set forth herein uses a transposase removal fluid to weaken the interaction between the transposase enzyme 12 and the tagmented DNA by employing a chelator molecule under conditions where it binds to and sequesters the divalent cation cofactor from the active site of the transposase enzyme 12. In doing so, the transposase removal fluid dissociates the transposase enzyme 12 from the tagmented DNA, making the DNA accessible for processing by downstream enzymes.

An example of the method disclosed herein includes exposing a deoxyribonucleic acid sample to tagmentation in the presence of a tagmentation buffer including a divalent cation cofactor and a transposase enzyme 12, thereby generating a tagmented DNA fragment complex; adding a transposase removal fluid to the tagmented DNA fragment complex, the transposase removal fluid: including a chelator of the divalent cation cofactor at a weight ratio that is at least 1:1 with the divalent cation cofactor, and having a pH ranging from 8 to 9; incubating the tagmented DNA fragment complex in the transposase removal fluid at a temperature of at least 55° C. for at least about 60 seconds, whereby the transposase enzyme 12 dissociates from a tagmented DNA fragment of the tagmented DNA fragment complex. Some examples of the method further include washing the transposase removal fluid and the dissociated transposase enzyme 12 from the tagmented DNA fragment. Other examples of the method further include adding a reagent to append an adapter to the 3′ end of the tagmented DNA fragment.

The transposase removal fluid includes the chelator of the divalent cation cofactor (used in tagmentation); a buffer agent; and water. In some examples, the transposase removal fluid also includes a salt.

The chelator is a reagent that sequesters the cation used for tagmentation, for example, Mg²⁺, Co²⁺, or the like. As such, the chelator of the transposase removal fluid will depend upon the divalent cation cofactor used in tagmentation. In one example, the divalent cation cofactor is Mg²⁺; and the chelator is selected from the group consisting of ethylenediaminetetraacetic acid (EDTA) and a diketoacid antiretroviral compound. In another example, the divalent cation cofactor is Co²⁺; and the chelator is selected from the group consisting of an aza-crown-ether compound, N-acetyl-cysteine, a porphyrin, and a crown ether. When the chelator is the aza-crown-ether compound, the aza-crown-ether compound may be 1,4,7,10-tetraazacyclododecane tetrahydrochloride. When the chelator is the crown ether, the crown ether may be selected from the group consisting of 15-crown-5 and 18-crown-6. When the chelator is the diketoacid antiretroviral compound, the diketoacid antiretroviral compound may be selected from the group consisting of 2-hydroxy-4-oxo-4-thiophen-2-yl-but-2-enoic acid, 4-thien-2-yl-2,4-dioxobutanoic acid, and 2,4-dioxo-4-phenyl butanoic acid, which are used as integrase inhibitors. It is believed that other viral integrase inhibitors may be used, such as those that have a common structural motif with the transposase being removed. When the chelator is the porphyrin, the porphyrin may be vitamin B12 without the cobalt.

The amount of the chelator in the transposase removal fluid will depend upon the amount of the divalent cation cofactor to be used in tagmentation and upon whether a salt is included in the transposase removal fluid. The chelator is present in a weight ratio that is at least 1:1 with the divalent cation cofactor to be used in tagmentation. When the salt is not included in the transposase removal fluid, the transposase removal can be achieved by including an excess of the chelator relative to the divalent cation cofactor. The excess chelator is effective in promoting the chelation of the divalent cation cofactor away from the active site of the protein. In these examples, the transposase removal fluid includes the chelator in a weight ratio ranging from about 3:1 to about 10:1 with the divalent cation cofactor to be used in tagmentation. When the salt is included in the transposase removal fluid, the transposase removal can be achieved with a smaller amount of the chelator (compared to when the salt is not included). The salt may bind to the phosphate backbone of the tagmented DNA, rendering it unable to act like a ligand for the divalent cation cofactor and making the divalent cation cofactor more susceptible to chelation. In these examples, the transposase removal fluid includes the chelator in a weight ratio ranging from 1:1 to about 3:1 with the divalent cation cofactor to be used in tagmentation. In one example of the transposase removal fluid that includes the salt, the weight ratio of the chelator in the transposase removal fluid to the divalent cation cofactor used in tagmentation is 1.2:1.

When included, the salt of the transposase removal fluid may be an inorganic salt selected from the group consisting of a sodium salt, a potassium salt, and a lithium salt. Example sodium salts include sodium chloride, sodium sulfate, and sodium carbonate; an example potassium salt includes potassium chloride; and an example lithium salt includes lithium chloride.

When included, the salt in the transposase removal fluid is present at a concentration of at least 75 mM. In one example, the salt is present at a concentration of 150 mM.

Any suitable buffer agent may be used in the transposase removal fluid, such as tris(hydroxymethyl)aminomethane (Tris buffer), Tris hydrochloride (Tris-HCl), Tris acetate salt, etc.

The concentration of the buffer agent in the transposase removal fluid ranges from about 5 mM to about 100 mM.

The pH of the transposase removal fluid ranges from 8 to 9.

The transposase removal fluid may be part of a kit that also includes a tagmentation buffer. In one example, the kit includes the tagmentation buffer, which includes water, an optional co-solvent (e.g., dimethylformamide), a divalent cation cofactor for the transposase 12, and a buffer agent; and the transposase removal fluid, which includes water, a chelator of the divalent cation cofactor in the tagmentation buffer at a weight ratio that is at least 1:1 with the divalent cation cofactor, and a buffer agent. In some examples, the transposase removal fluid of the kit also includes a salt at a concentration of at least 75 mM. In an example of the tagmentation buffer, the optional co-solvent may be present in an amount up to about 11%, the metal co-factor may be present in a concentration ranging from about 1.5 mM to about 5.5 mM, and the buffer agent may be present in a concentration ranging from about 5 mM to about 12 mM. The balance of the tagmentation buffer is water (e.g., deionized water).

In one example of the method, the divalent cation cofactor (in the tagmentation buffer) is Mg²⁺ and the chelator (in the transposase removal fluid) is EDTA at a weight ratio, relative to Mg²⁺, of at least 1:1, e.g., 1.2:1, in a buffer that contains a salt, for example NaCl, at a concentration of at least 75 mM, e.g., 150 mM, and at a pH of at least 8.0, e.g., ranging from 8.6 to 9. This example of the transposase removal fluid can be incubated with the tagmented DNA complex at a temperature of at least 55° C., e.g., 60° C., for a duration of at least 60 seconds.

In another example of the method, the divalent cation cofactor (in the tagmentation buffer) is Mg²⁺ and the chelator (in the transposase removal fluid) is a diketoacid antiretroviral compound at a weight ratio, relative to Mg²⁺, of at least 1:1 in a buffer that contains a salt, for example NaCl, at a concentration of at least 75 mM, e.g., 150 mM, and at a pH of at least 8.0, e.g., ranging from 8.6 to 9. This example of the transposase removal fluid is incubated with the tagmented DNA complex at a temperature of at least 55° C., e.g., 60° C., for a duration of at least 60 seconds.

In still another example of the method, the divalent cation cofactor (in the tagmentation buffer) is Co²⁺ and the chelator (in the transposase removal fluid) is Cyclen (i.e., 1,4,7,10-Tetraazacyclododecane tetrahydrochloride). In this example, the Co²⁺ is used at a concentration of at least 1.5 mM, e.g., 5 mM, and the Cyclen chelator is present at a weight ratio, relative to Co²⁺, of at least 1:1 (e.g., 1.5 mM or 7.5 mM). The Cyclen is present in a buffer that contains a salt, for example NaCl, at a concentration of at least 75 mM, e.g., 150 mM, and at a pH of at least 8.0, e.g., ranging from 8.6 to 9. This example of the transposase removal fluid is incubated with the tagmented DNA complex at a temperature of at least 55° C., e.g., 60° C., for a duration of at least 60 seconds.

The time for incubating the tagmented DNA complex in the transposase removal fluid may range from about 1 minute to about 5 minutes, and the temperature for this incubation period is about 60° C. It is to be understood that a temperature gradient may be used throughout the incubation period, where the temperature is ramped up to 60° C. and is maintained at 60° C. for at least one minute.

After tagmentation and transposase removal as described herein, and prior to downstream processing that appends the 3′ ends of the fragments, the method may further include adding cations (e.g., Mg²⁺) that support the activity of enzymes, for examples, ligases and polymerases, used to append that 3′ adaptor to the tagmented DNA. The addition of these cations compensates for the prior sequestration of tagmentation cations to enable transposase removal.

To further illustrate the present disclosure, examples are given herein. It is to be understood that these examples are provided for illustrative purposes and are not to be construed as limiting the scope of the present disclosure.

### NON-LIMITING WORKING EXAMPLES

### Example 1

This example illustrates results from two comparative methods (experiment A and experiment B) for removing the Tn5 transposase enzyme after tagmentation. For each of these experiments, the tagmentation buffer included water, 10% dimethyl formamide (DMF), 5 mM magnesium acetate, and 10 mM tris acetate salt, pH 7.6. As such Mg²⁺ was the divalent cation cofactor.

In the workflow for experiment A: DNA was first tagmented, appending the transferred strand of the adaptor to the 5′ ends of the tagmented DNA, and then treated with water (not sodium dodecyl sulfate (SDS)), thus keeping the Tn5 bound to the tagmented DNA. The tagmented DNA complex was washed and subjected to PCR (including high temperature heating which may dissociate the Tn5) to append adaptor sequences to the 3′ ends of the tagmented DNA.

In the workflow for experiment, B: DNA was first tagmented, appending the transferred strand of the adaptor to the 5′ ends of the tagmented DNA, and then was treated with SDS to denature the Tn5 enzyme, thus dissociating it from the tagmented DNA complex. The SDS was washed away and the tagmented DNA fragments were exposed to PCR to append adaptor sequences to the 3′ ends of the tagmented DNA.

The tagmented DNA fragments from experiments A and B were exposed to a solid phase reversible immobilization (SPRI) technique to reversibly bind the tagmented DNA as a non-size selecting cleanup process. In this process, SPRI beads were added to the respective samples at 1.8 times the volume of the PCR buffer, and were incubated at room temperature for about 5 minutes. The tagmented DNA bound to the SPRI beads was pelleted, washed in ethanol followed by ethanol removal, resuspended in a buffer, and pelleted again. Some of the supernatant was analyzed using a TapeStation. The size profile and yield of the tagmented DNA fragments from experiments A and B were measured using the TapeStation. The results for both experiments A and B are shown in FIG. 4. For a library prepared with fully removed Tn5, the expected size profile (based in part on the transposome complex concentration used) included fragments ranging from 100 base pairs (bp) to 4,000 bp. At higher or lower transposome complex concentrations, the expected size range may shift. The results in FIG. 4 illustrate that SDS (line B) is more effective in removing Tn5 from the tagmented product complex than the high temperature heating used in PCR (line A). In contrast to the results for the SDS Tn5 removal (line B), the data for Tn5 removal without SDS (line A) indicated a greatly reduced yield and a skew in insert size profiles.

### Example 2

This example illustrates results from one comparative method (experiment A) and one example method (experiment B) for removing the Tn5 transposase enzyme after tagmentation. For each of these experiments, the tagmentation buffer included water, 10% DMF, 5 mM magnesium acetate, and 10 mM tris acetate salt, pH 7.6. As such, Mg²⁺ was the divalent cation cofactor. Incubation for tagmentation was performed at 55° C. for 5 minutes.

In the workflow for experiment A: DNA was first tagmented, appending the transferred strand of the adaptor to the 5′ ends of the tagmented DNA. Tn5 was removed by adding 1% SDS and incubating for 5 minutes at 25° C. The SDS was washed away in a wash buffer and the sample was processed under the PCR-cycling conditions with EPM as the polymerase and buffer base.

In the workflow for experiment B: DNA was first tagmented, appending the transferred strand of the adaptor to the 5′ ends of the tagmented DNA. The supernatant was discarded and Tn5 was removed by adding an example of the transposase removal fluid disclosed herein. In experiment B, the transposase removal fluid included 100 mM Tris-HCl, pH 8.6, 100 mM NaCl, and 0.6 mM EDTA. The tagmented DNA complex was incubated in the transposase removal fluid for 1 minute at 60° C. Following a wash in the wash buffer, the sample was processed under PCR-cycling conditions with EPM as the polymerase and buffer base.

The tagmented DNA fragments from experiments A and B were exposed to the same SPRI technique described in Example 1 to reversibly bind the tagmented DNA as a non-size selecting cleanup process, except that the size profile and yield of the tagmented DNA fragments from experiments A and B in this example were measured using a Bioanalyzer. The results (respectively labeled A and B) are shown in FIG. 5. The data for experiment A and experiment B demonstrates equivalent yield and insert size profiles between the conventional SDS approach (line A) and the cation-chelation approach (line B) for removing Tn5 from its product complex. As such, the data in FIG. 5 demonstrates that a transposase removal fluid comprising EDTA and salt in a buffer at a high pH, in combination with heat, is sufficient to replace SDS as a reagent for dissociating the Tn5 enzyme from a tagmented DNA complex.

### Example 3

This example illustrates results from a replicated comparative method (experiments A and B) and a replicated example method (experiments C and D) for removing the Tn5 transposase enzyme after tagmentation. For each of these experiments, the tagmentation buffer included water, 10% DMF, 5 mM magnesium acetate, and 10 mM tris acetate salt, pH 7.6. As such Mg²⁺ was the divalent cation cofactor. Incubation for tagmentation was performed at 55° C. for 5 minutes.

In the workflow for experiments A and B: DNA was first tagmented, appending the transferred strand of the adaptor to the 5′ ends of the tagmented DNA. Tn5 was removed by adding 1% SDS and incubating for 5 minutes at 25° C. The SDS was washed away in a wash buffer and the sample was processed under the PCR-cycling conditions with EPM as the polymerase and buffer base.

In the workflow for experiments C and D: DNA was first tagmented, appending the transferred strand of the adaptor to the 5′ ends of the tagmented DNA. The Tn5 was removed by adding an example of the transposase removal fluid disclosed herein. In experiments C and D, the transposase removal fluid included 100 mM Tris-HCl, pH 8.6, 100 mM NaCl, and 6 mM EDTA. The tagmented DNA complex was incubated in the transposase removal fluid for 1 minute at 60° C. Following a wash in the wash buffer, the sample was processed under PCR-cycling conditions with EPM as the polymerase and buffer base.

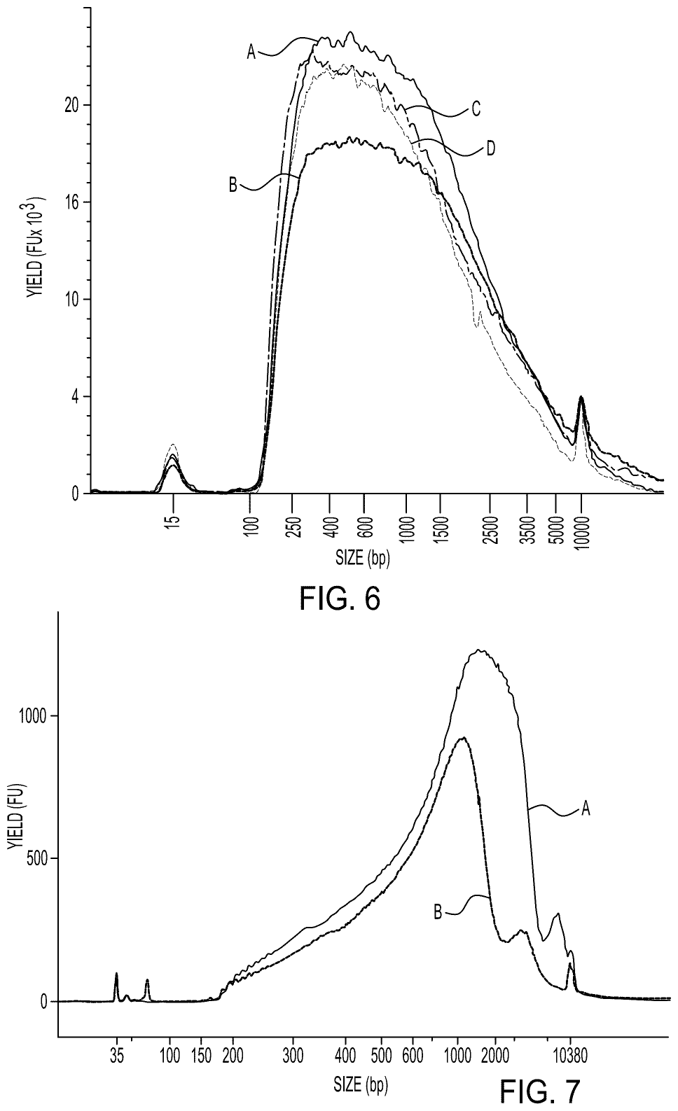

The tagmented DNA fragments from experiments A through D were exposed to the same SPRI technique described in Example 1 to reversibly bind the tagmented DNA as a non-size selecting cleanup process. The size profile and yield results are shown in FIG. 6. The results in FIG. 6 illustrate that the transposase removal fluid comprising EDTA and salt in a buffer at a high pH, in combination with heat, can be applied directly to a tagmented reaction without removing the supernatant (which was performed in Example 2), and can effectively dissociate Tn5 from the tagmented DNA complex. As such, the transposase removal fluid may be introduced without performing a first wash that exchanges the tagmentation buffer.

### Example 4

This example illustrates results from a comparative method (experiment A) and an example method (experiment B) for removing the Tn5 transposase enzyme after tagmentation. For each of these experiments, the tagmentation buffer included water, 4% DMF, 2 mM magnesium acetate, and 4 mM tris acetate salt, pH 7.6. As such Mg²⁺ was the divalent cation cofactor. Incubation for tagmentation was performed at 55° C. for 5 minutes.

In the workflow for experiment A: DNA was first tagmented, appending the transferred strand of the adaptor to the 5′ ends of the tagmented DNA. Tn5 was removed by adding 1% SDS and incubating for 5 minutes at 25° C. The SDS was washed away in a wash buffer and the sample was processed under Extension-Ligation and PCR-cycling conditions with a ligase, non-strand displacing polymerase and Q5 polymerase in a buffer base.

In the workflow for experiment B: DNA was first tagmented, appending the transferred strand of the adaptor to the 5′ ends of the tagmented DNA. The Tn5 was removed by adding an example of the transposase removal fluid disclosed herein. In experiment B, the transposase removal fluid included 140 mM Tris, pH 8.8, 100 mM KCl, and 1.8 mM EDTA. The tagmented DNA complex was incubated in the transposase removal fluid for 1 minute at 60° C. The sample was processed under Extension-Ligation and PCR-cycling conditions with a ligase, non-strand displacing polymerase and Q5 polymerase in a buffer base with 2.4 mM Magnesium.

The tagmented DNA fragments from experiments A and B were exposed to the same SPRI technique described in Example 1 to reversibly bind the tagmented DNA as a non-size selecting cleanup process, except that some of the supernatant was analyzed using a Bioanalyzer. The size profile and yield results are shown in FIG. 7. These results illustrate that the transposase removal fluid comprising EDTA and salt in a buffer at a high pH, in combination with heat, can be applied directly to a tagmented reaction and can dissociate Tn5 from the tagmented DNA complex. These results also indicate that following the dissociation of Tn5, a composition comprising a ligase, non-displacing polymerase and Q5 polymerase in a base buffer containing magnesium, can be added directly to the reaction without removing the supernatant (e.g., without exchanging the buffer), and the enzymes are able to function.

### Example 5

This example illustrates results from a positive control method (experiment A), a first comparative method (experiment B), an example method (experiment C), and a second comparative method (experiment D) for removing the Tn5 transposase enzyme after tagmentation. For experiments A and D, the tagmentation buffer included water, 10% DMF, 5 mM magnesium acetate, and 10 mM tris acetate salt, pH 7.6. In these experiments, Mg²⁺ was the divalent cation cofactor. For experiments B and C, the tagmentation buffer included water, 10% dimethyl formamide (DMF), 2.5 mM cobalt, and 25 mM Tris-HCl, pH 7.6. In these experiments, Co²⁺ was the divalent cation cofactor. For all of the experiments, incubation for tagmentation was performed at 55° C. for 5 minutes.

In the workflow for experiment A: DNA was first tagmented using the Mg²⁺ tagmentation buffer described in this example, appending the transferred strand of the adaptor to the 5′ ends of the tagmented DNA. Tn5 was removed by adding 1% SDS and incubating for 5 minutes at 25° C. The SDS was washed away in a wash buffer and the sample was processed under the PCR-cycling conditions with EPM as the polymerase and buffer base.

In the workflow for experiment B: DNA was first tagmented using the Co²⁺ tagmentation buffer described in this example, appending the transferred strand of the adaptor to the 5′ ends of the tagmented DNA. Tn5 was removed by adding 1% SDS and incubating for 5 minutes at 25° C. The SDS was washed away in a wash buffer and the sample was processed under the PCR-cycling conditions with EPM as the polymerase and buffer base.

In the workflow for experiment C: DNA was first tagmented using the Co²⁺ tagmentation buffer described in this example, appending the transferred strand of the adaptor to the 5′ ends of the tagmented DNA. The Tn5 was removed by adding an example of the transposase removal fluid disclosed herein. In experiment C, the transposase removal fluid included 100 mM Tris, pH 8.6, 150 mM KCl, and 5 mM Cyclen. The tagmented DNA complex was incubated in the transposase removal fluid for 1 minute at 60° C. Following a wash in the wash buffer, the sample was processed under PCR-cycling conditions with EPM as the polymerase and buffer base.

In the workflow for experiment D: DNA was first tagmented using the Mg²⁺ tagmentation buffer described in this example, appending the transferred strand of the adaptor to the 5′ ends of the tagmented DNA. The Tn5 was removed by adding an example of the transposase removal fluid disclosed herein. In experiment D, the transposase removal fluid included 100 mM Tris, pH 8.6, 150 mM KCl, and 5 mM Cyclen. The tagmented DNA complex was incubated in the transposase removal fluid for 1 minute at 60° C. Following a wash in the wash buffer, the sample was processed under PCR-cycling conditions with EPM as the polymerase and buffer base.

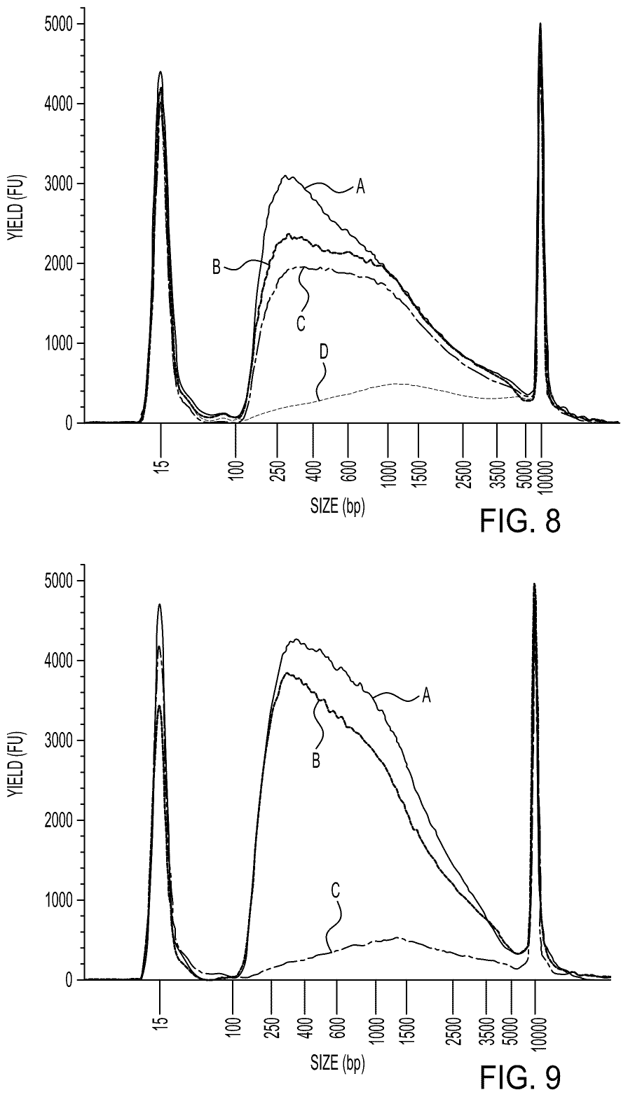

The tagmented DNA fragments from experiments A through D were exposed to the same SPRI technique described in Example 1 to reversibly bind the tagmented DNA as a non-size selecting cleanup process. The size profile and yield results are shown in FIG. 8. Line B in FIG. 8 illustrates that the Co²⁺ cation can substitute for Mg²⁺ in the catalytic mechanism of Tn5. Line C in FIG. 8 illustrates that the Co²⁺ cation can substitute for Mg²⁺ in the catalytic mechanism of Tn5 and that a composition containing Cyclen that chelates Co²⁺ can serve to disassociate Tn5 from the tagmented DNA complex, resulting in a suitable library template size profile and yield following PCR. Line D in FIG. 8 illustrates that Cyclen is ineffective at chelating Mg²⁺ from the active site of the enzyme. Overall, the results in FIG. 8 demonstrate that the transposase removal fluid comprising Cyclen and salt in a buffer at a high pH, in combination with heat, is sufficient to replace SDS as a reagent for dissociating the Tn5 enzyme from a tagmented DNA complex when Co²⁺ cations are used as the cofactor.

### Example 6

This example illustrates results from an example method (experiment A), positive control method (experiment B), and a comparative method (experiment C) for removing the Tn5 transposase enzyme after tagmentation. For each of these experiments, the tagmentation buffer included water, 10% DMF, 5 mM magnesium acetate, and 10 mM tris acetate salt, pH 7.6. As such Mg²⁺ was the divalent cation cofactor. Incubation for tagmentation was performed at 55° C. for 5 minutes.

In the workflow for experiment A: DNA was first tagmented, appending the transferred strand of the adaptor to the 5′ ends of the tagmented DNA. The Tn5 was removed by adding an example of the transposase removal fluid disclosed herein. In experiment A, the transposase removal fluid included 500 mM Tris, pH 8.6, 100 mM NaCl, and 15 mM 2-hydroxy-4-oxo-4-thiophen-2-yl-but-2-enoic acid integrase inhibitor. The tagmented DNA complex was incubated in the transposase removal fluid for 1 minute at 60° C. Following a wash in the wash buffer, the sample was processed under PCR-cycling conditions with EPM as the polymerase and buffer base.

In the workflow for experiment B: DNA was first tagmented, appending the transferred strand of the adaptor to the 5′ ends of the tagmented DNA. Tn5 was removed by adding 1% SDS and incubating for 5 minutes at 25° C. The SDS was washed away in a wash buffer and the sample was processed under the PCR-cycling conditions with EPM as the polymerase and buffer base.

In the workflow for experiment C: DNA was first tagmented, appending the transferred strand of the adaptor to the 5′ ends of the tagmented DNA, and then treated with water (not the transposase removal fluid or the sodium dodecyl sulfate (SDS)), thus keeping the Tn5 bound to the tagmented DNA. The tagmented DNA complex was washed and subjected to PCR (including high temperature heating which may dissociate the Tn5) to append adaptor sequences to the 3′ ends of the tagmented DNA.

The tagmented DNA fragments from experiments A through C were exposed to the same SPRI technique described in Example 1 to reversibly bind the tagmented DNA as a non-size selecting cleanup process. The size profile and yield results are shown in FIG. 9. These results demonstrate that compounds designed to inhibit integrase enzymes can be used to chelate a cation from the active site of a Tn5 transposome product complex, thus dissociating the Tn5 from the DNA and enabling a sequencing library to be generated.

### Example 7

This example illustrates results from a positive control method (experiment A and B), and an example method (experiment C and D) for removing the Tn5 transposase enzyme after tagmentation. For experiments A and B, the tagmentation buffer included water, 2 mM magnesium acetate, and 10 mM tris-HCl, pH 7.5. In these experiments, Mg²⁺ was the divalent cation cofactor. For experiments C and D, the tagmentation buffer included water and 1.25 mM cobalt. In these experiments, Co²⁺ was the divalent cation cofactor. For all of the experiments, incubation for tagmentation was performed at 37° C. for 15 minutes.

In the workflow for experiments A and B: DNA was first tagmented using the Mg²⁺ tagmentation buffer described in this example, appending the transferred strand of the adaptor to the 5′ ends of the tagmented DNA. Tn5 was removed by adding 1% SDS and incubating for 5 minutes at 25° C. The SDS was washed away in a wash buffer and the sample was processed under the PCR-cycling conditions with EPM as the polymerase and buffer base.

In the workflow for experiments C and D: DNA was first tagmented using the Co²⁺ tagmentation buffer described in this example, appending the transferred strand of the adaptor to the 5′ ends of the tagmented DNA. The Tn5 was removed by adding an example of the transposase removal fluid disclosed herein. In experiments C and D, the transposase removal fluid included 95 mM Tris, pH 8.8, 75 mM KCl, and 3.75 mM Cyclen. The tagmented DNA complex was incubated in the transposase removal fluid for 1 minute at 65° C. Following a wash in the wash buffer, the sample was processed under PCR-cycling conditions with EPM as the polymerase and buffer base.

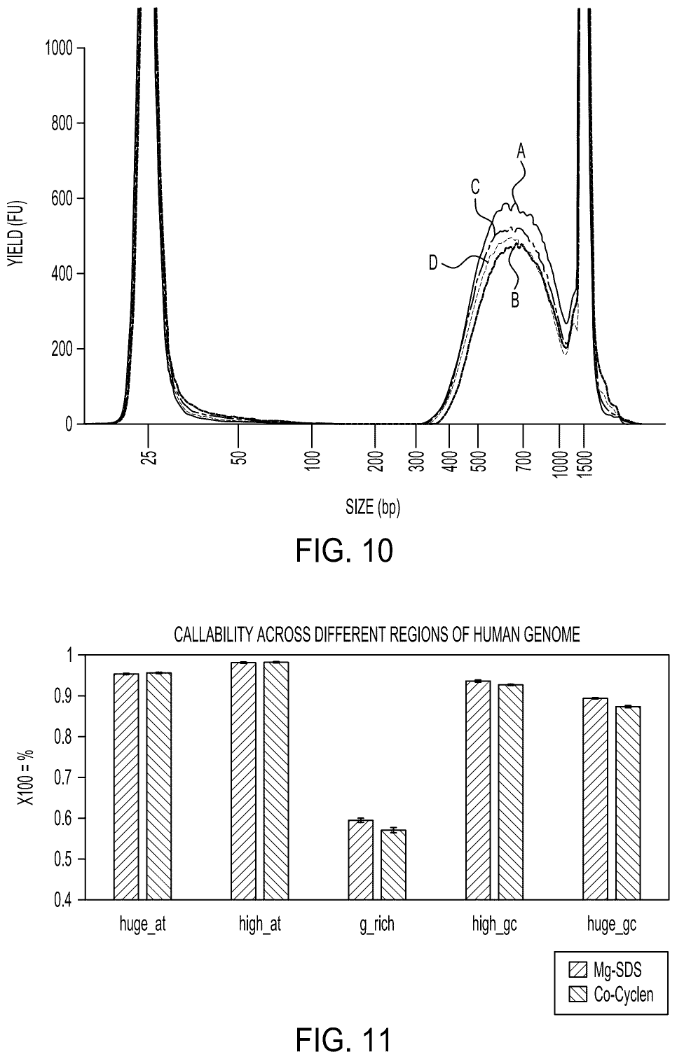

The tagmented DNA fragments from experiments A through D were exposed to a solid phase reversible immobilization (SPRI) technique to reversibly bind the tagmented DNA as a size selecting cleanup process. In this process, SPRI beads were added to the respective samples at 0.5 times the volume of the PCR buffer, and were incubated at room temperature for about 5 minutes. The tagmented DNA bound to the SPRI beads was pelleted. The supernatant was then transferred to a new tube and additional SPRI beads were added at a 0.12 times ration compared to the supernatant volume. The samples were incubated at room temperature for about 5 minutes. The SPRI beads were then pelleted, washed in ethanol followed by ethanol removal, resuspended in a buffer and then pelleted again. Some of the supernatant was analyzed using a TapeStation. The size profile and yield results are shown in FIG. 10. The peaks of the lines (A-D) in FIG. 10 are not reproduced herein, as these portions of the lines were marker peaks used to align the sample data, but were not part of the sample data. Lines A and B in FIG. 10 show the size profiles and yields obtained from the control workflow using Mg²⁺ in the Tagmentation reaction and SDS to dissociate the Tn5. Lines C and D in FIG. 10 illustrate that the Co²⁺ cation can substitute for Mg²⁺ in the catalytic mechanism of Tn5, and that a composition containing Cyclen that chelates Co²⁺ can serve to disassociate Tn5 from the tagmented DNA complex, resulting in a suitable library template size profile and yield following PCR. Overall, the results in FIG. 10 demonstrate that the transposase removal fluid comprising Cyclen and salt in a buffer at a high pH, in combination with heat, is sufficient to replace SDS as a reagent for dissociating the Tn5 enzyme from a tagmented DNA complex when Co²⁺ cations are used as the cofactor.

These comparative and example samples were then sequenced on a NOVASEQ™ 6000 instrument according to the customer user guide and the run was analyzed and downsampled to 30× coverage using the Fluente: Dragen Downsample v1.0 App in Basespace. The data in FIG. 11 shows comparable performance in the accuracy or callability of the comparative and example samples across regions of the human genome with different AT and GC contents. The AT and GC contents referenced in FIG. 11 are defined below in Table 1.

TABLE 1

| Identifier in FIG. 11 | Definition |
|---|---|
| huge_at | At least 100 base pairs (bp) within a DNA |
| | fragment, where AT is equal to or over 85% of |
| | the bases sequenced |
| high_at | At least 100 bp within a DNA fragment, where |
| | AT is equal to or over 75% of the bases |
| | sequenced |
| g_rich | At least 30 bp within a DNA fragment, where |
| | G is equal to or over 80% of the bases |
| | sequenced |
| high_gc | At least 100 bp within a DNA fragment, where |
| | GC is equal to or over 75% of the bases |
| | sequenced |
| huge_gc | At least 100 bp within a DNA fragment, where |
| | GC is equal to or over 85% of the bases |
| | sequenced |

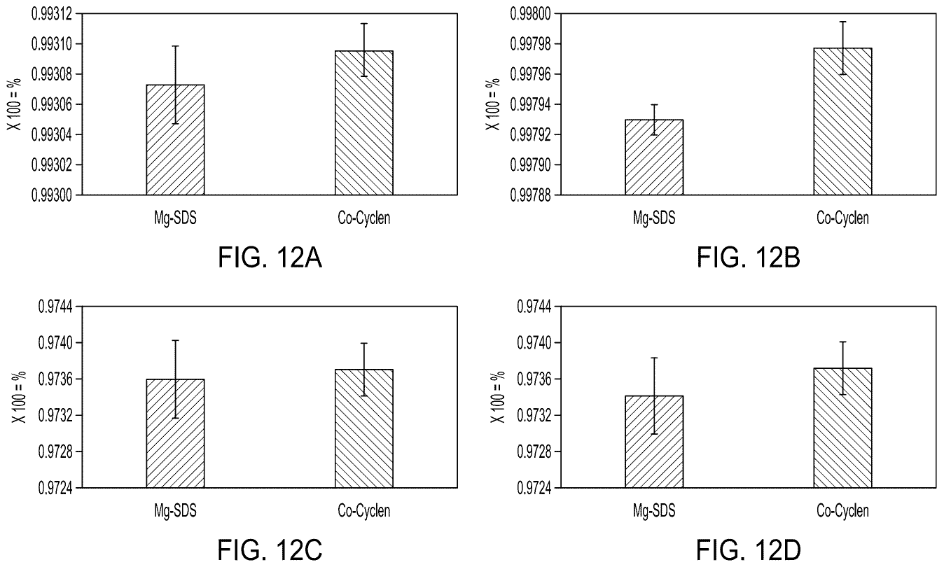

During sequencing, a single nucleotide polymorphism (SNP) changes a single nucleotide in a DNA sequence and an indel incorporates or removes one or more nucleotides. These secondary metrics were also collected, and are shown in FIG. 12A through FIG. 12D. In these graphs, precision refers to accuracy and is calculated as the ratio of:

[# of True Positive Calls/(# of True Positive Calls+# of False Positive Calls)]

and recall refers to sensitivity and is calculated as the ratio of:

[# of True Positive Calls/(# of True Positive Calls+# of False Negative Calls)]

The results in FIG. 12A through FIG. 12D illustrate equivalent performance of the comparative and example methods to both detect and accurately call both SNPs and Indels compared to the known truth set for the NA12878 human platinum genome sample.

The results in FIG. 11 and FIG. 12A through FIG. 12D demonstrate that the transposase removal fluid does not deleteriously affect downstream sequencing.

### Representative Items

Item 1. A method, comprising:
exposing a deoxyribonucleic acid sample to tagmentation in the presence of a tagmentation buffer including a divalent cation cofactor and a transposase enzyme, thereby generating a tagmented DNA fragment complex;
adding a transposase removal fluid to the tagmented DNA fragment complex, the transposase removal fluid:
   including a chelator of the divalent cation cofactor at a weight ratio that is at least 1:1 with the divalent cation cofactor; and
   having a pH ranging from 8 to 9; and
incubating the tagmented DNA fragment complex in the transposase removal fluid at a temperature of at least 55° C. for at least about 60 seconds, whereby the transposase enzyme dissociates from a tagmented DNA fragment of the tagmented DNA fragment complex.
   Item 2. The method as defined in item 1, wherein:
the divalent cation cofactor is Mg²⁺; and
the chelator is selected from the group consisting of ethylenediaminetetraacetic acid and a diketoacid antiretroviral compound.
   Item 3. The method as defined in item 1, wherein:
the divalent cation cofactor is Co²⁺; and
the chelator is selected from the group consisting of an aza-crown-ether compound, N-acetyl-cysteine, a porphyrin, and a crown ether.
   Item 4. The method as defined in item 3, wherein:
the chelator is the aza-crown-ether compound; and
the aza-crown-ether compound is 1,4,7,10-tetraazacyclododecane tetrahydrochloride.
   Item 5. The method as defined in item 3, wherein:
the chelator is the crown ether; and
the crown ether is selected from the group consisting of 15-crown-5 and 18-crown-6.
   Item 6. The method as defined in item 1, further comprising a salt at a concentration of at least 75 mM.
   Item 7. The method as defined in item 6, wherein the salt is an inorganic salt selected from the group consisting of a sodium salt, a potassium salt, and a lithium salt.
   Item 8. The method as defined in item 1, further comprising washing the transposase removal fluid and the dissociated transposase enzyme from the tagmented DNA fragment.
   Item 9. The method as defined in item 1, further comprising introducing reagents to the tagmented DNA fragment to append adapter sequences to a 3′ end of the tagmented DNA fragment.
   Item 10. A transposase removal fluid, comprising:
a chelator of a divalent cation cofactor;
a buffer agent; and
water.
   Item 11. The transposase removal fluid as defined in item 10, wherein:
the divalent cation cofactor is Mg²⁺; and
the chelator is selected from the group consisting of ethylenediaminetetraacetic acid and a diketoacid antiretroviral compound.
   Item 12. The transposase removal fluid as defined in item 10, wherein:
the divalent cation cofactor is Co²⁺; and
the chelator is selected from the group consisting of an aza-crown-ether compound, N-acetyl-cysteine, a porphyrin, and a crown ether.
   Item 13. The transposase removal fluid as defined in item 12, wherein:
the chelator is the aza-crown-ether compound; and
the aza-crown-ether compound is 1,4,7,10-tetraazacyclododecane tetrahydrochloride.
   Item 14. The transposase removal fluid as defined in item 12, wherein:
the chelator is the crown ether; and
the crown ether is selected from the group consisting of 15-crown-5 and 18-crown-6.
   Item 15. The transposase removal fluid as defined in item 10, further comprising a salt at a concentration of at least 75 mM, wherein the salt is an inorganic salt selected from the group consisting of a sodium salt, a potassium salt, and a lithium salt.
   Item 16. A kit, comprising:
a tagmentation buffer including:
   water;
   a co-solvent;
   a divalent cation cofactor for a transposase enzyme; and
   a buffer agent; and
a transposase removal fluid including:
   water;
   a chelator of the divalent cation cofactor in the tagmentation buffer at a weight ratio that is at least 1:1 with the divalent cation cofactor in the tagmentation buffer; and
   a buffer agent.
      Item 17. The kit as defined in item 16, wherein the transposase removal fluid further comprises a salt at a concentration of at least 75 mM.

### ADDITIONAL NOTES

It should be appreciated that all combinations of the foregoing concepts and additional concepts discussed in greater detail below (provided such concepts are not mutually inconsistent) are contemplated as being part of the inventive subject matter disclosed herein. In particular, all combinations of claimed subject matter appearing at the end of this disclosure are contemplated as being part of the inventive subject matter disclosed herein. It should also be appreciated that terminology explicitly employed herein that also may appear in any disclosure incorporated by reference should be accorded a meaning most consistent with the particular concepts disclosed herein.

Reference throughout the specification to “one example”, “another example”, “an example”, and so forth, means that a particular element (e.g., feature, structure, and/or characteristic) described in connection with the example is included in at least one example described herein, and may or may not be present in other examples. In addition, it is to be understood that the described elements for any example may be combined in any suitable manner in the various examples unless the context clearly dictates otherwise.

While several examples have been described in detail, it is to be understood that the disclosed examples may be modified. Therefore, the foregoing description is to be considered non-limiting.

1. A method, comprising:
exposing a deoxyribonucleic acid sample to tagmentation in the presence of a tagmentation buffer including a divalent cation cofactor and a transposase enzyme, thereby generating a tagmented DNA fragment complex;
adding a transposase removal fluid to the tagmented DNA fragment complex, the transposase removal fluid:
including a chelator of the divalent cation cofactor at a weight ratio that is at least 1:1 with the divalent cation cofactor; and
having a pH ranging from 8 to 9; and
incubating the tagmented DNA fragment complex in the transposase removal fluid at a temperature of at least 55° C. for at least about 60 seconds, whereby the transposase enzyme dissociates from a tagmented DNA fragment of the tagmented DNA fragment complex.

2. The method as defined in claim 1, wherein:
the divalent cation cofactor is Mg²⁺; and
the chelator is selected from the group consisting of ethylenediaminetetraacetic acid and a diketoacid antiretroviral compound.

3. The method as defined in claim 1, wherein:
the divalent cation cofactor is Co²⁺; and
the chelator is selected from the group consisting of an aza-crown-ether compound, N-acetyl-cysteine, a porphyrin, and a crown ether.

4. The method as defined in claim 3, wherein:
the chelator is the aza-crown-ether compound; and
the aza-crown-ether compound is 1,4,7,10-tetraazacyclododecane tetrahydrochloride.

5. The method as defined in claim 3, wherein:
the chelator is the crown ether; and
the crown ether is selected from the group consisting of 15-crown-5 and 18-crown-6.

6. The method as defined in claim 1, wherein the transposase removal fluid further comprises a salt at a concentration of at least 75 mM, and wherein the salt is optionally an inorganic salt selected from the group consisting of a sodium salt, a potassium salt, and a lithium salt.

7. The method as defined in claim 1, further comprising washing the transposase removal fluid and the dissociated transposase enzyme from the tagmented DNA fragment.

8. The method as defined in claim 1, further comprising introducing reagents to the tagmented DNA fragment to append adapter sequences to a 3′ end of the tagmented DNA fragment.

9. A transposase removal fluid, comprising:
a chelator of a divalent cation cofactor;
a buffer agent; and
water.

10. The transposase removal fluid as defined in claim 9, wherein:
the divalent cation cofactor is Mg²⁺; and
the chelator is selected from the group consisting of ethylenediaminetetraacetic acid and a diketoacid antiretroviral compound.

11. The transposase removal fluid as defined in claim 9, wherein:
the divalent cation cofactor is Co²⁺; and
the chelator is selected from the group consisting of an aza-crown-ether compound, N-acetyl-cysteine, a porphyrin, and a crown ether.

12. The transposase removal fluid as defined in claim 11, wherein:
i) the chelator is the aza-crown-ether compound; and
the aza-crown-ether compound is 1,4,7,10-tetraazacyclododecane tetrahydrochloride; or
ii) the chelator is the crown ether; and
the crown ether is selected from the group consisting of 15-crown-5 and 18-crown-6.

13. The transposase removal fluid as defined in claim 9, further comprising a salt at a concentration of at least 75 mM, wherein the salt is optionally an inorganic salt selected from the group consisting of a sodium salt, a potassium salt, and a lithium salt.

14. A kit, comprising:
a tagmentation buffer including:
water;
a co-solvent;
a divalent cation cofactor for a transposase enzyme; and
a buffer agent; and
a transposase removal fluid including:
water;
a chelator of the divalent cation cofactor in the tagmentation buffer at a weight ratio that is at least 1:1 with the divalent cation cofactor in the tagmentation buffer; and
a buffer agent.

15. The kit as defined in claim 14, wherein the transposase removal fluid further comprises a salt at a concentration of at least 75 mM.

16. The kit as defined in claim 14, wherein:
i) the divalent cation cofactor is Mg²⁺; and
the chelator is selected from the group consisting of ethylenediaminetetraacetic acid and a diketoacid antiretroviral compound; or
ii) the divalent cation cofactor is Co²⁺; and
the chelator is selected from the group consisting of an aza-crown-ether compound, N-acetyl-cysteine, a porphyrin, and a crown ether.
